(19) United States
(12) Patent Application Publication
(10) Pub. No.: US 2022/0409387 A1
(43) Pub. Date: Dec. 29, 2022

(54) METHODS AND SYSTEMS FOR FACILITATING TREATMENT OF LUMBAR DEGENERATIVE DISC DISEASE BASED ON TOTAL NUCLEUS REPLACEMENT USING MAGNETIC SPHERICAL BEADS

(71) Applicants: OLUWATODIMU RICHARD RAJI, SAN FRANCISCO, CA (US); DIMITRIY KONDRASHOV, SAN FRANCISCO, CA (US); JEREMI MICHAEL LEASURE, CARLSBAD, CA (US)

(72) Inventors: OLUWATODIMU RICHARD RAJI, SAN FRANCISCO, CA (US); DIMITRIY KONDRASHOV, SAN FRANCISCO, CA (US); JEREMI MICHAEL LEASURE, CARLSBAD, CA (US)

(21) Appl. No.: 17/848,128

(22) Filed: Jun. 23, 2022

Related U.S. Application Data
(60) Provisional application No. 63/214,067, filed on Jun. 23, 2021.

(51) Int. Cl. A61F 2/44; A61F 2/46

(52) U.S. Cl. CPC A61F 2/442; A61F 2/4611; A61F 2002/444; A61F 2002/30079

(57) ABSTRACT

A system and method for facilitating the treatment of lumbar degenerative disc disease. The method comprising making an incision posterolaterally along an annulus fibrosus of a specimen, removing nucleus pulposus from the specimen with tissue removing tools, and pushing magnetic spherical beads into a nuclear area within the specimen. The system comprises the specimen with the nuclear area, a plurality of magnetic spherical beads, tissue removal tools, a hollow cannula and a non-magnetic rod. The hollow cannula and non-magnetic rod configured for pushing the magnetic spherical beads within the nuclear area after incision.

### FIELD OF THE INVENTION

The present invention relates generally to surgery. More specifically, the present invention is methods and systems for facilitating treatment of lumbar degenerative disc disease based on total nucleus replacement using magnetic spherical beads.

### BACKGROUND OF THE INVENTION

The field of surgery is technologically important to several industries, business organizations, and/or individuals.

Degenerative Disc Disease (DDD) is a ubiquitous diagnosis in the aging population. It is a major cause of pain and disability worldwide. DDD may be accompanied by axial pain, radiculopathy, myelopathy, spinal instability, deformity, and specific imaging findings. DDD often results in the development of debilitating back pain. This leads to patients' activity levels becoming pain-limited, contributing to the development of comorbidities, including weight gain, diabetes, heart disease, and dependence on pain medication.

Surgical approaches to DDD include spinal decompression, discectomy, fusion, or disc replacement. The current standard of care, spinal fusion across two or more vertebrae, prevents motion, increases stability, and is theorized to decrease pain. Major criticisms of spinal fusion surgery include accelerated degeneration of discs above or below the level of fusion as well as alteration of kinematics at adjacent segments. Therefore, there is a need for a method and device to restore function and normal spine kinematics.

For years, disc replacements have been proposed and studied as a potential solution. Total disc replacement procedures involve the complete removal of the intervertebral disc to replace with an artificial disc. This attempts to maintain and/or restore disc height, retain the motion between the vertebrae, and indirectly decompress the neural elements by restoring the disk height and indirectly decompressing the neuroforamina. While a total disc replacement surgery allows for the motion between vertebrae, this may cause abnormal motion and stress to the posterior elements at the treated levels, especially with the older ball-and-socket designs. The challenges of current total nucleus replacement, often known as partial disc replacement, include migration or expulsion of the implant, and subsidence.

Therefore, there is a need for improved methods and systems for facilitating treatment of lumbar degenerative disc disease based on total nucleus replacement using magnetic spherical beads that may overcome one or more of the above-mentioned problems and/or limitations.

### SUMMARY OF THE INVENTION

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

According to some embodiments, a method for facilitating treatment of lumbar degenerative disc disease based on total nucleus replacement using magnetic spherical beads is disclosed. Further, the method may include making a 5 mm incision posterolaterally along an annulus fibrosus. Further, the method may include inserting rongeurs and curettes through the incision. Further, the method may include removing about 2 grams of nucleus pulposus based on the inserting. After nucleotomy testing, the method may include pushing a predetermined number of magnetic neodymium spheres (or beads) into a nuclear space for the treatment using a 5 mm hollow cannula and a non-magnetic rod Further, the magnetic spherical beads (or balls) may be characterized by a diameter of 3 mm. Further, the magnetic spherical beads may be used to increase the surface area where the implant construct contacts the endplates. Further, the magnetic spherical beads have the added benefit of drawing together, potentially preventing extrusion through the defect (on incision) in the annulus fibrosus created during implantation. Further, the beads may increase stability during flexion and extension; evident in a range of motion values after bead insertion being comparable to intact values.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 illustrates L2-L3 lumbar vertebrae of a specimen, in accordance with some embodiments.

FIG. 2 illustrates L2-L3 lumbar vertebrae of the specimen with tissue removal tools inserted into an incision, in accordance with some embodiments.

FIG. 3 illustrates L2-L3 lumbar vertebrae of the specimen receiving magnetic spherical beads using a hollow cannula and non-magnetic rod, in accordance with some embodiments.

FIG. 4 illustrates L2-L3 lumbar vertebrae of the specimen with the magnetic spherical beads, in accordance with some embodiments.

FIG. 5 is a flowchart of a method for facilitating treatment of lumbar degenerative disc disease, in accordance with some embodiments.

FIG. 6 is a flowchart of a method of performing nucleotomy testing, in accordance with some embodiments.

FIG. 7 is a flowchart of a method of pushing a predetermined number of magnetic spherical beads into a nuclear space, in accordance with some embodiments.

FIG. 8 illustrates multidirectional bending flexibility results of L2/L3 specimens for each study group in three planes of rotation motion, in accordance with some embodiments.

FIG. 9 illustrates L2-L3 lumbar vertebrae receiving the magnetic spherical beads using the 5 mm hollow cannula and the non-magnetic rod, in accordance with some embodiments.

### DETAIL DESCRIPTIONS OF THE INVENTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of methods and systems for facilitating treatment of lumbar degenerative disc disease based on total nucleus replacement using magnetic spherical beads, embodiments of the present disclosure are not limited to use only in this context.

The present disclosure describes methods and systems for facilitating treatment of lumbar degenerative disc disease based on total nucleus replacement using magnetic spherical beads. Total nuclear replacements have been developed as an alternative less invasive procedure seeking to correct the effects of DDD with less change to the biomechanics of the spinal column. Rather than replacing the entire disc, a nuclear replacement selectively replaces the nucleus pulposus. Maintaining the annulus fibrosus provides structural support for the injected biomaterials, however, the defect created in the annulus during the implantation procedures provides a possible site for the biomaterial extrusion.

Lumbar Degenerative Disc Disease is a potential cause of spinal instability. Surgical techniques such as arthrodesis or arthroplasty are implemented to restore stability to the spine. Both a fusion and a disc replacement are substantially invasive. A lumbar disk replacement is typically done via retroperitoneal or Transperitoneal approach. With an increasing number of symptomatic levels, the disk replacement becomes incrementally more invasive, with a potential for significant blood loss, injury to great vessels, injury to abdominal viscera, weakening of abdominal wall, etc. There is no minimally invasive way to perform a disk replacement either at one or multiple levels. Further, the method may include less-invasive total nucleus replacement using magnetic spherical beads in a cadaver-based multidirectional bending flexibility model. Further, the ability of the beads to restore stability to a spine segment may be assessed. Further, it may be hypothesized that the beads would increase stability during flexion and extension motions.

Three fresh-frozen, human cadaveric L2-L3 segments were used. Further, multidirectional flexibility tests in six directions, under intact, nucleotomy and bead insertion conditions may be performed. Further, the ranges of motions, the neutral zone, and the elastic zone may be calculated.

There may be statistically significantly more flexion-extension range of motion, neutral zone and elastic zone observed upon nucleotomy than during intact condition. Bead insertion statistically significantly reduced the neutral zone observed after nucleotomy, during flexion, and extension loading. Further, the disclosed total nucleus replacement is capable of restoring the stability to spine motion segments during flexion and extension motions, using adjustments to their neutral zones.

Further, the disclosed Total Nucleus Replacement (TNR) to address the limitations of the previous biomaterial implantation options. Further, the disclosed method may include utilizing metallic, magnetic spherical beads as TNR. Further, functional biomechanical testing is carried out to establish how well this type of construct maintains the motion characteristics of the native disc. Further, it may be hypothesized that the implanted beads may increase stability during flexion and extension; evident in a range of motion values after bead insertion being comparable to intact values.

Further, the three frozen cadaveric spinal segments were harvested. The specimens were grossly examined for the absence of spinal pathology before inclusion. After examination, the L2-L3 spinal segments were isolated and stripped of the soft tissue, leaving ligamentous structures, facet structures, the nucleus pulposus, and annulus fibrosus intact. The L2-L3 levels may be chosen due to the specimen availability. The specimens may be then frozen until potting for testing.

Specimen Preparation:

Before potting, the specimens may include wood screws inserted to increase adherence to the epoxy. 3 to 4 wood screws may be drilled halfway into the cranial and caudal aspects of the L2 and L3 vertebral bodies, respectively. The specimen may be then potted using epoxy in a large silicone fixture for the caudal aspect and a smaller metallic fixture for the cranial aspect. The specimens may be aligned using the cylindrical shape of the spine to fit the cylindrical shape of the fixture. The shelves of the potting may not be parallel since the lumbar region of the spine from which the specimens were harvested has a natural lordotic curvature.

The floating moment ring, which is used to impart pure moment forces on the specimens were attached cranially with screws, and the base may be secured to linear bearings situated on the test frame using cap screws. Two pedicle screws may be attached to the anterior aspect of the vertebral bodies, with one screw per spinal level to later mount optical tracking markers. The screws may be placed on opposite sides. During testing, specimens were covered with wetted gauze applied around the circumference of the intervertebral disc.

Optical Motion Tracking:

Two triangular test frame markers (Optotrak marker triads) may be attached to the specimen, one per pedicle screw, facing laterally and with their LEDs facing the camera. The movement of the markers may be captured by an optoelectronic system (Optotrak 3020; Northern Digital, Inc.). The accuracy of the Optotrak 3020 optical active-marker system has been reported to have a bias of 0.05° and 0.03 mm, with repeatability limits of 0.67° and 0.29 mm.

Biomechanical Testing:

The biomechanical test and Optotrak calibration were run through the MFlexWin software. All specimens underwent a low-speed lumbar spine test in three planes: flexion/extension, axial rotation, and lateral bending. The ranges of motion may be recorded in flexion and extension, left and right lateral bending, as well as left and right axial rotation.

For each condition, the loading may be applied to a single functional spinal unit in the superior-inferior orientation. Loading was applied via a servo-hydraulic test frame (858 Mini Bionix II; MTS) through a custom fixture for pure moment loading attached to the superior L2 vertebra. A preload of 7.5 Nm may be applied three times for 45 seconds with 15 seconds of rest in between preloads for each specimen. During testing, each specimen underwent a steadily increasing load from 0-7.5 Nm with 1.5 Nm increment increases every 45 seconds. Data may be collected through the MFlexWin (custom) software and stored on a personal computer before being run through the DextrWin (custom) software to extract the axis of rotation and translational data. Specimens may be tested in the following conditions: intact, after nucleotomy, and after bead insertion.

Creation of Nucleotomy Specimens, and Bead Insertion:

After intact testing, a nucleotomy may be performed on intact specimens. A 5 mm incision was made posterolaterally along the annulus fibrosus. Rongeurs and curettes may be inserted through the incision to remove about 2 grams of the nucleus pulposus. After nucleotomy testing, a 5 mm hollow cannula and non-magnetic rod were used to push 80 magnetic neodymium spheres into the nuclear space for the treatment testing.

Data Analysis:

Range of Motion (ROM), Elastic Zone (EZ), and Neutral Zone (NZ) may be calculated for each specimen at each condition. The neutral zone was defined as the difference in degrees in the zero-load state between each paired moment, after three cycles of preconditioning for each load (ie, flexion-extension, right-left axial, right-left lateral). The range of motion may be defined as the displacement in degrees from the maximum moment to respect the maximum moment for the paired load taken at the final loading cycle. The elastic zone may be defined as the range of motion minus the neutral zone.

The intact data may be analyzed using paired t-tests between the intact specimens in flexion/extension, lateral bending, and axial rotation. The data comparing between treatment conditions may be analyzed using a one-way analysis of variance (ANOVA) with posthoc comparisons using Tukey-Kramer HSD (JMP, Version 15. SAS Institute Inc., Cary, N.C., 1989-2021). For statistical purposes, the data population being sampled from was assumed to be normal; however, with only three samples normality is unlikely, thus, results should not be taken as proof of statistical significance, but a trend towards significance.

Data for each functional spinal unit was compared between the 3 conditions (intact, nucleotomy, bead insertion) for each pure moment. P-values less than 0.05 may be considered statistically significant. All data is displayed as mean±standard deviation unless otherwise stated.

Results:

Further, the three specimens aged 52-57 from two female and one male cadaver underwent the above-mentioned testing.

| Current | | | Height | Weight | |
|---|---|---|---|---|---|
| state | Gender | Age | (Inches) | (Lbs) | BMI |
| L2-L3 | F | 52 | 64 | 130 | 22 |
| L2-L3 | M | 52 | 76 | 265 | 32.3 |
| L2-L3 | F | 57 | 62 | 185 | 33.8 |

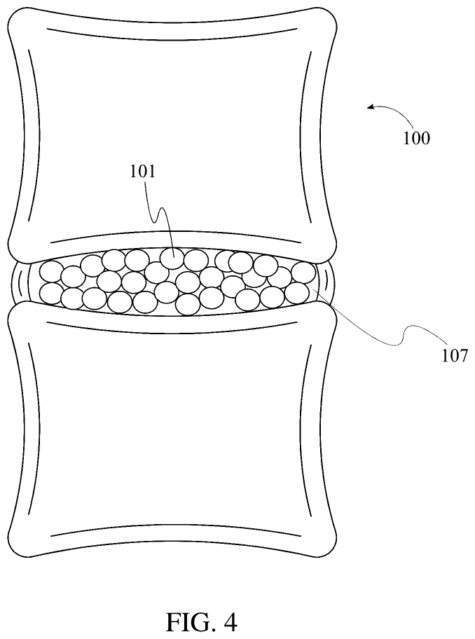

Further, the ranges of rotational motion in each of the three anatomical planes as well as their neutral and elastic zones are displayed in FIG. 4. Axial rotation generated the lowest range of motion in all study groups (intact 1.96°±0.86°, nucleotomy 3.75°±0.79°, replacement 2.82°±1.13°, p<0.028), and there were no significant differences in range of motion between flexion-extension (intact 4.83°±0.62°, nucleotomy 9.72°±1.53°, replacement 8.61°±1.76°) and lateral bending motions (intact 6.59°±1.76°, nucleotomy 10.04°±1.20°, replacement 9.48°±2.36° (p≥0.220). When compared against the intact condition, nucleotomy generated significant range of motion increase during flexion-extension (intact 4.83°±0.62° vs nucleotomy 9.72°±1.53°, p=0.012), there was also similar increase during lateral bending (intact 6.59°±1.76° vs nucleotomy 10.04°±1.20°, p=0.132), and axial rotation (intact 1.96°±0.86° vs nucleotomy 3.75°±0.79°, p=0.128), but these were not significant. The neutral zone may be also increased in all loading planes, although only flexion-extension (intact 0.37°±0.33° vs nucleotomy 2.82°±0.67°, p=0.006) was statistically significant. In all loading planes, the elastic zone increased upon nucleotomy, but may be only statistically significant during flexion-extension (intact 4.56°±0.62° vs nucleotomy 6.90°±0.99°, p=0.050).

Upon nucleus replacement after nucleotomy, range of motion decreased in flexion/extension (nucleotomy 9.72°±1.53° vs replacement 8.61°±1.76°, p=0.617) and axial rotation (nucleotomy 3.75°±0.79° vs replacement 2.82°±1.13°, p=0.493) across all specimens, although these reductions may not be statistically significant. Reduction may be seen in the neutral zone in flexion/extension (nucleotomy 2.82°±0.67° vs replacement 1.06°±0.73°, p=0.027) that may be statistically significant, as well as axial rotation, although this may not be statistically significant (nucleotomy 0.41°±0.23° vs replacement 0.23°±0.10°, p=0.392). Across all specimens, the elastic zone increased during flexion/extension (nucleotomy 6.90°±0.99° vs replacement 7.55°±1.12°, p=0.687), but reduced during axial rotation (nucleotomy 3.34°±0.57° vs replacement 2.59°±1.04°, p=0.533), none of these changes may be not statistically significant.

Research has been continuous regarding the ideal materials and methods of nucleus replacement in patients with DDD as the perfect solution to current nucleus replacement complications has yet to be determined. With the existing nucleus replacement options, subsidence through the endplates, extrusion through annular defects, and adjacent segment changes remain recurrent complications. With the disclosed novel total nucleus replacement, these concerns may be addressed by introducing the proof of concept of utilizing neodymium magnetic balls as a nucleus implant prototype. Further, the disclosed method increased spinal stability during flexion/extension as indicated by the significant reduction in the neutral zone upon nucleus replacement. Improved upon the original single Fernstrom Ball technique, the neodymium magnetic balls may be used to increase the surface area where the implant construct contacts the endplates. In theory, this physiologic distribution of load and contact pressure could prevent endplate changes and avoid subsidence. The fluidity of the balls allows them to conform to the dead space created by the removal of nucleus pulposus and to distribute themselves as needed throughout the space. With movement, the localized pressure points between the implant and endplates will change, preventing areas of bone yielding or bone resorption in high strain and lower strain areas, respectively. By design, the ultimate construct of the magnetic balls is customizable by altering the number of balls implanted as well as the size of the individual balls themselves. This theoretically allows the surgeon to calculate the required number and diameter of balls based on both the amount of nucleus pulposus removed and targeted disc height restoration. By continuing to collect data on the resulting outcome measures of ROM, NZ, and EZ based on these input variables, the calculation may be standardized to determine the optimal implant construct.

Both stainless steel and cobalt-chromium balls have been used in previous literature with similar goals in mind. Neodymium magnetic balls have the added benefit of drawing together, potentially preventing extrusion through the defect (or the incision) in the annulus fibrosus created during implantation. Preceding studies indicate that the success rate of repairs of the annulus fibrosus by means of adhesive bonding or sutures has been minimal though some find the use of Annular Closure Devices (ACD) more promising. Further, the disclosed method facilitates removal of the hurdle of ineffective annulus repair; the attraction between the balls keeps them within the target area while allowing the rearrangement among them required during the range of motion of the spinal column.

To similarly address complications from extrusion through the annular defect, Zengerle et al implemented the use of an ACD in conjunction with a novel collagen-based nucleus implant. Further, they were able to perform comparable cyclic loading tests without evidence of implant extrusion from within the annulus fibrosus. Further, the disclosed method may facilitate accomplishing this without the longer procedure time and device-related complications of the additional step of ACD implementation. Other studies utilized finite element analyses to test their implant concept properties, but without comparable cadaveric testing, it is difficult to compare outcomes. Reitmaier discusses the importance of restoring the interface between the nucleus implant and the surrounding structures for proper restoration of the native biomechanical function. Unlike typical hydrogel nucleus replacements, the disclosed novel method does not allow for interdigitation of the magnetic balls with the adjacent annulus and endplates as their macroscopic size does not provide ideal conditions for facilitating a cellular migration.

As with all studies, our study had limitations involved in the process. Due to the cost and availability of cadaver specimens, only three specimens may be tested, leading to low power. Restoration of the disc height and intradiscal pressure may not be measured outcomes. The bone quality of the donor vertebrae may not be measured. Future studies may be required to determine the ratio of the amount of nucleus pulposus removed vs. a number of magnetic balls implanted, the ideal size of individual magnetic balls, and how best to customize these properties to optimize the stiffness of the implant construct. While there may be no observed migration or expulsion over the course of our testing, further tests are required to definitively assess this.

This study utilized a cadaver-based biomechanical model to evaluate the potential viability of a novel and less invasive total nucleus replacement using magnetic spherical beads. The aim may be to assess the ability of the beads to restore stability to the spine segment. It was hypothesized that the beads may increase stability during flexion and extension; evident in a range of motion values after bead insertion being comparable to intact values. Bead insertion statistically significantly reduced the neutral zone observed after nucleotomy, during flexion and extension loading. These initial results suggest that magnetic spherical beads may be a suitable replacement for the nucleus pulposus.

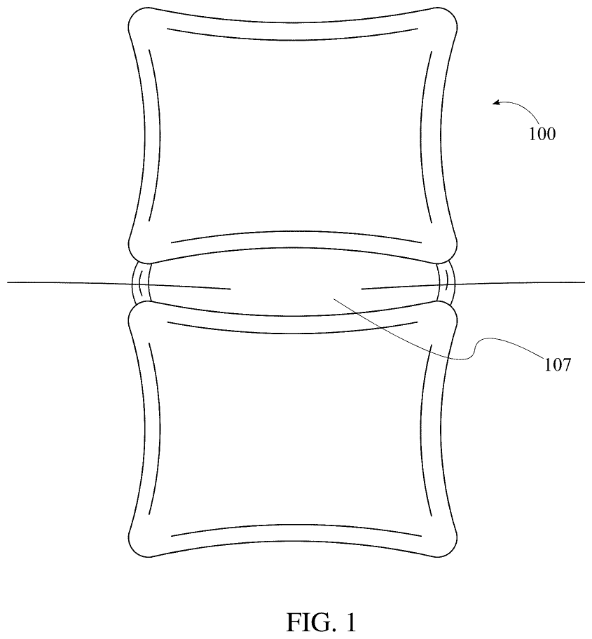

FIG. 1 illustrates L2-L3 lumbar vertebrae of a specimen 100, in accordance with some embodiments.

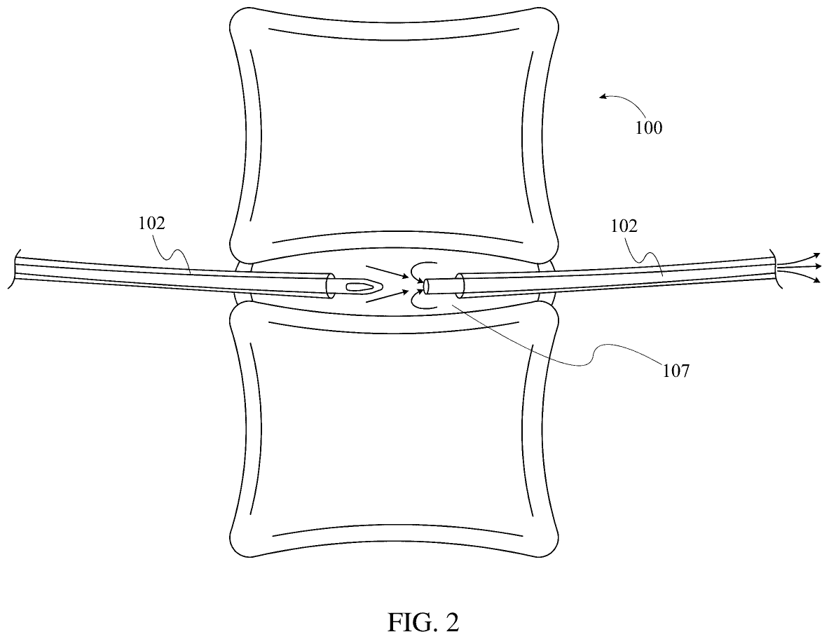

FIG. 2 illustrates L2-L3 lumbar vertebrae of the specimen 100 with tissue removal tools 102 inserted into an incision, in accordance with some embodiments.

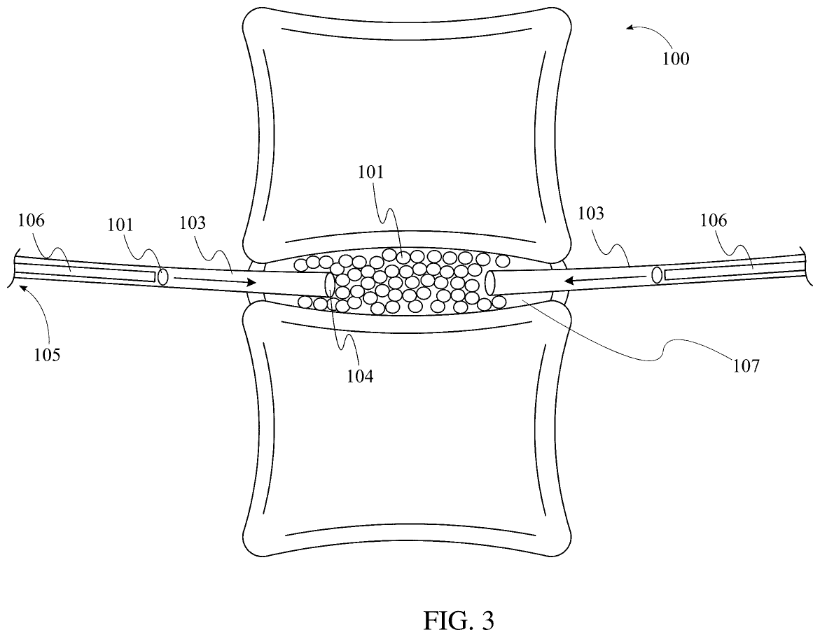

FIG. 3 illustrates L2-L3 lumbar vertebrae of the specimen 100 receiving magnetic spherical beads 101 using a hollow cannula 103 and non-magnetic rod 106, in accordance with some embodiments.

FIG. 4 illustrates L2-L3 lumbar vertebrae of the specimen 100 with the magnetic spherical beads 101, in accordance with some embodiments.

The system of the present invention generally includes, the specimen 100 comprising a nuclear space 107, the plurality of magnetic spherical beads 101, at least one tissue removal tool 102, the hollow cannula 103, and the non-magnetic rod. In the preferred embodiment, the hollow cannula 103 is 5 mm in diameter, having a first end 104 and an open end 105 and being configured to accept both the plurality of magnetic spherical beads 101 and the non-magnetic rod 106. The at least one tissue removal tool 102 of the system is preferably rongeurs and curettes, though other tools may be used. In the preferred embodiment, the plurality of magnetic spherical beads 101 are magnetic neodymium spheres coated in a hydrogel or similar polymer.

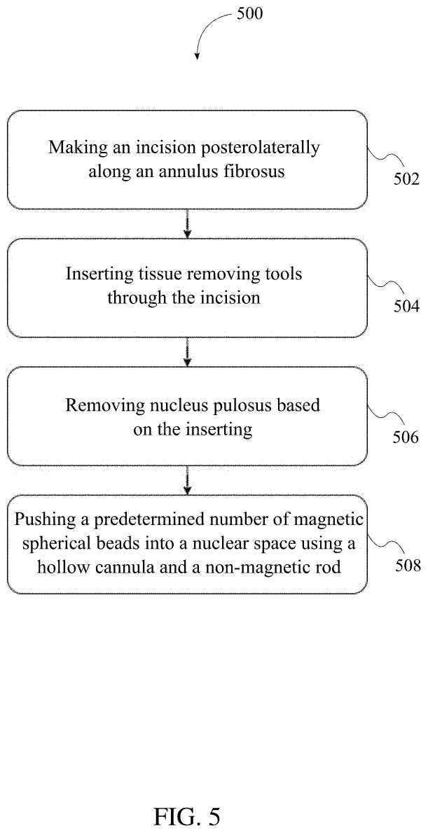

The method of the present invention for facilitating the treatment of lumbar degenerative disc disease based on total nucleus replacement using magnetic spherical beads is disclosed. In FIG. 5, the method 500 for facilitating treatment of lumbar degenerative disc disease is shown. The method 500 may include performing nucleotomy on a specimen 100 (lumbar vertebrae such as L2 and L3). Further, the method 500 may include, at 502, making a 5 mm incision posterolaterally along an annulus fibrosus. Further, the method 500 may include, at 504, inserting tissue removal tools 102, such as rongeurs and curettes, through the incision. Further, the method 500 may include, at 506 removing about 2 grams of nucleus pulposus based on the inserting. The method 500 may include, at 508, pushing a predetermined number of magnetic spherical beads 101 (or beads) into a nuclear space 107 for the treatment using the 5 mm hollow cannula 103 and the non-magnetic rod 106 (as shown in FIG. 2 and FIG. 3). Further, the magnetic spherical beads 101 may be characterized by a diameter of 3 mm. Further, the magnetic spherical beads 101 (or balls) may be used to increase the surface area where the implant construct contacts the endplates. Further, the magnetic spherical beads are preferably magnetic neodymium spheres 101 having the added property of drawing together, potentially preventing extrusion through the defect (or the incision) in the annulus fibrosus created during implantation. Further, the magnetic spherical beads may be coated with a hydrogel or similar polymer to reduce the stiffness of the magnetic spherical beads, allow for compliance and compression of the plurality of magnetic spherical beads, and aid in sealing the incision or preventing extrusion of the plurality of magnetic spherical beads through the incision. Further, the disclosed method facilitates removal of the hurdle of ineffective annulus repair; the attraction between the magnetic spherical beads 101 keeps them within the target area while allowing the rearrangement among them required during a range of motion of the spinal column. Further, the magnetic spherical beads 101 may increase stability during flexion and extension, evident in a range of motion values after bead insertion being comparable to intact values. Further, the magnetic spherical beads 101 may be a suitable replacement for the nucleus pulposus. Further, the disclosed method may facilitate restoring the stability to spine motion segments during flexion and extension motions, by means of adjustments to their neutral zones.

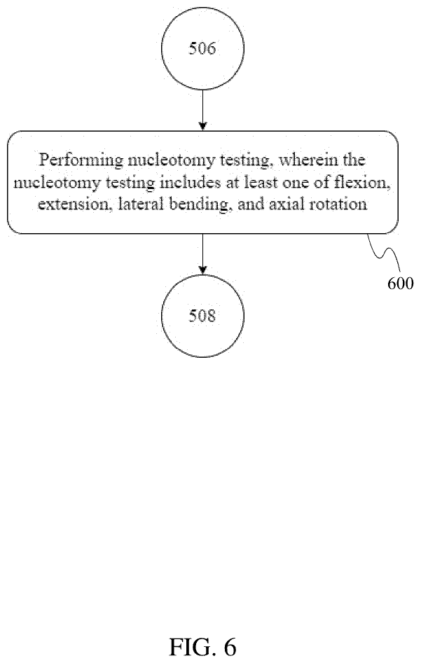

In some embodiments of the present invention, as shown in FIG. 6, the method 500 may further include, at 600, performing nucleotomy testing after the step 506 of removing nucleus pulposus from the specimen and before the step 508 of pushing magnetic spherical beads. The performing nucleotomy testing 600, may include at least one of flexion, extension, lateral bending, and axial rotation of the specimen following nucleotomy.

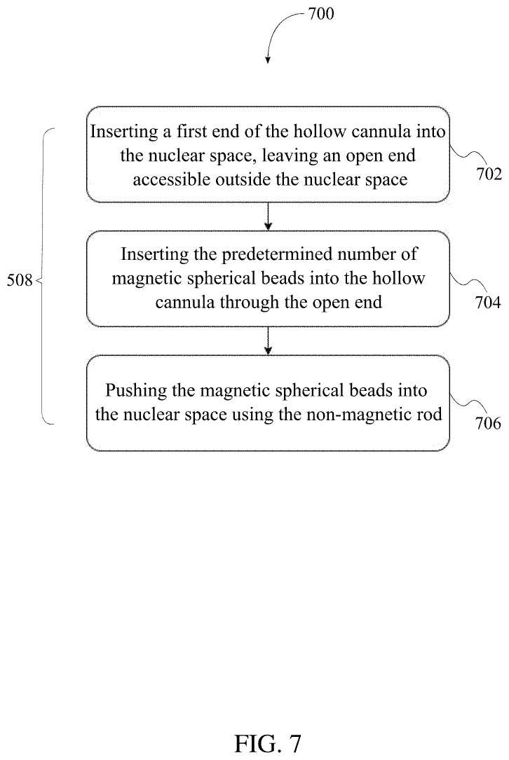

In some embodiments of the present invention, as shown in FIG. 7, the step 508 of pushing a predetermined number of magnetic spherical beads 101 (or balls) into a nuclear space 107 may comprise the submethod 700. The submethod 700 may include, at 702, inserting a first end 104 of the hollow cannula 103 into the nuclear space 107, leaving an open end 105 accessible outside the nuclear space 107. Further, the submethod 700 may include, at 704, inserting the predetermined number of magnetic spherical beads 101 into the hollow cannula 103 through the open end 105. Further, the submethod 700 may include, at 706, pushing the magnetic spherical beads 101 into the nuclear space 107 using the non-magnetic rod 106. This submethod 704 may be done by pushing the magnetic spherical beads 101 through the hollow cannula 103 one at a time, in groups, or all at once. The illustration in FIG. 3 displays two hollow cannulas 103 and two non-magnetic rods 106, though the method may also be conducted with a single hollow cannula 103 and a single non-magnetic rod 106.

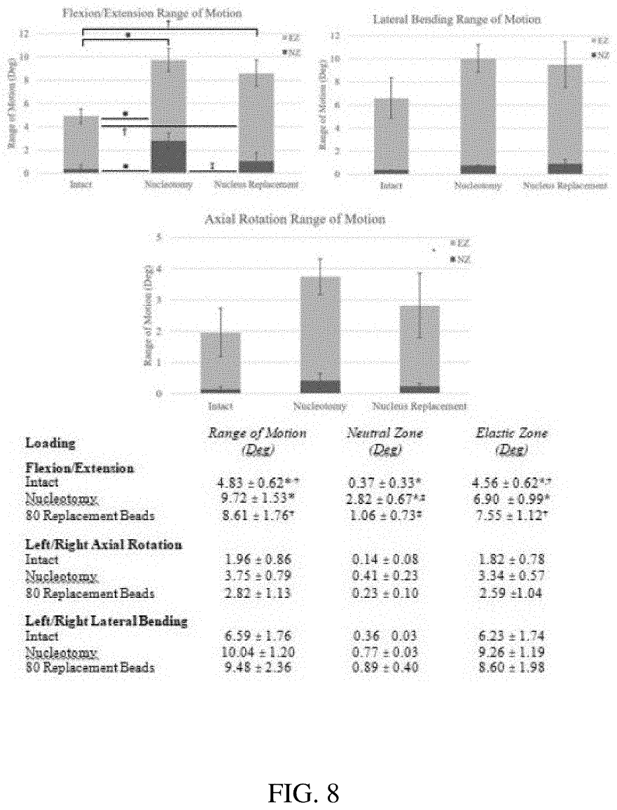

FIG. 8 illustrates multidirectional bending flexibility results of L2/L3 specimens for each study group in three planes of rotation motion, in accordance with some embodiments. Accordingly, all data are displayed as mean±one standard deviation. Range of motion was defined as the motion between the extents of loading in both directions. The neutral zone is the amount of displacement during passive resistance to loading and the elastic zone is the displacement during active resistance. The asterisk or note mark (*, †, ‡) indicates statistically significant differences between the designated pair for the given loading condition at p<0.05.

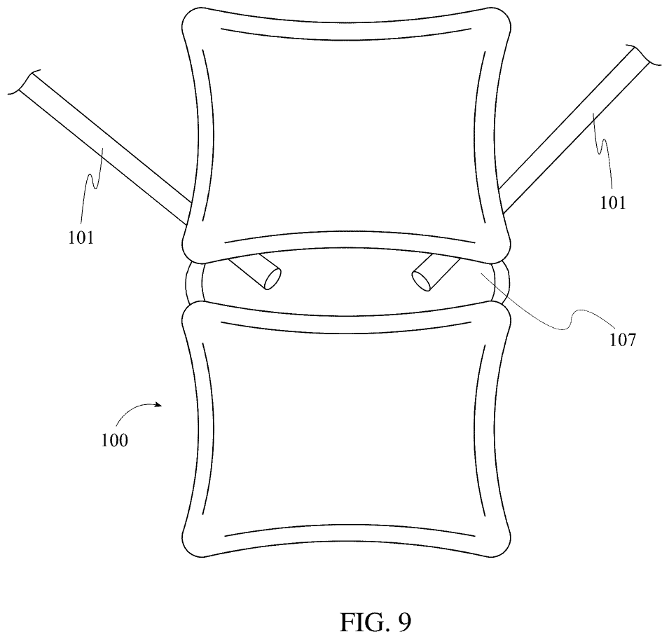

FIG. 9 illustrates L2-L3 lumbar vertebrae with the hollow cannula accessing the nuclear area by an alternative path through the bone, rather than through the annulus fibrosus.

It should be noted that FIG. 1-4, 9 illustrate the L2-L3 lumbar vertebrae as being enlarged in comparison to the surrounding anatomy as a way of more clearly illustrating the method of the present invention. Additionally, while the method of the present invention is best practiced in connection with the L2-L3 lumbar vertebrae, the systems, methods, and steps of the present invention are not limited to use with the L2-L3 lumbar vertebrae.

The terms “specimen” and “specimens” are used in some parts of this application to refer to spinal segments used in testing and research. However, these terms, as used in the descriptions of the drawings and the claims may also refer to patients and spinal segments undergoing the medical procedure outlined in the descriptions and claims.

Although the invention has been explained in relation to its preferred embodiment, it is to be understood that many other possible modifications and variations can be made without departing from the spirit and scope of the invention.

What is claimed is:

1. A method for facilitating the treatment of lumbar degenerative disc disease, comprising:
making an incision posterolaterally along an annulus fibrosus of a specimen;
inserting tissue removing tools through the incision;
removing nucleus pulposus based on the inserting; and
pushing a predetermined number of magnetic spherical beads into a nuclear space using a hollow cannula and a non-magnetic rod.

2. The method for facilitation the treatment of lumbar degenerative disc disease of claim 1, further comprising:
performing nucleotomy testing after the step of removing nucleus pulposus, wherein the nucleotomy testing includes at least one of flexion, extension, lateral bending, and axial rotation.

3. The method for facilitation the treatment of lumbar degenerative disc disease of claim 1, the step of pushing magnetic spherical beads into a nuclear space further comprising:
inserting a first end of the hollow cannula into the nuclear space, leaving an open end of the hollow cannula outside the specimen;
inserting the predetermined number of magnetic spherical beads into the hollow cannula through the open end; and
pushing the magnetic spherical beads into the nuclear space using the non-magnetic rod.

4. The method for facilitation the treatment of lumbar degenerative disc disease of claim 1, wherein the magnetic spherical beads are magnetic neodymium spheres configured to draw together, preventing extrusion of the magnetic spherical beads through the incision.

5. The method for facilitation the treatment of lumbar degenerative disc disease of claim 1, wherein the magnetic spherical beads are coated with a hydrogel to reduce the stiffness of the magnetic spherical beads, allow for compliance and compression of the plurality of magnetic spherical beads, and aid in sealing the incision or preventing extrusion of the plurality of magnetic spherical beads through the incision.

6. The method for facilitation the treatment of lumbar degenerative disc disease of claim 1, wherein the magnetic spherical beads are configured to rearrange during motion of the specimen.

7. The method for facilitation the treatment of lumbar degenerative disc disease of claim 1, wherein pushing the magnetic spherical beads into the nuclear space increases stability during flexion and extension of the specimen.

8. The method for facilitation the treatment of lumbar degenerative disc disease of claim 1, wherein the magnetic spherical beads are a suitable replacement for nucleus pulpous.

9. The method for facilitation the treatment of lumbar degenerative disc disease of claim 1, wherein pushing the magnetic spherical beads into the nuclear space facilitates restoring stability to spine motion segments of the specimen during flexion and extension motions, by means of adjustments to their neutral zones.

10. A system for facilitating the treatment of lumbar degenerative disc disease, comprising:
a specimen;
a plurality of magnetic spherical beads;
tissue removal tools;
a hollow cannula;
a non-magnetic rod;
the specimen further comprising a nuclear space; and
the plurality of magnetic spherical beads and the non-magnetic rod being configured to fit within the hollow cannula.

11. The system for facilitating the treatment of lumbar degenerative disc disease of claim 10, wherein the magnetic spherical beads are magnetic neodymium spheres configured to draw together, preventing extrusion of the magnetic spherical beads through the incision.

12. The system for facilitating the treatment of lumbar degenerative disc disease of claim 10, wherein the magnetic spherical beads are coated with a hydrogel to reduce the stiffness of the magnetic spherical beads, allow for compliance and compression of the plurality of magnetic spherical beads, and aid in sealing the incision or preventing extrusion of the plurality of magnetic spherical beads through the incision.

13. The system for facilitating the treatment of lumbar degenerative disc disease of claim 10, wherein the tissue removal tools are rongeurs and curettes.

14. The system for facilitating the treatment of lumbar degenerative disc disease of claim 10, further comprising:
the hollow cannula further comprising a first end and an open end;
the hollow cannula having a 5 mm diameter opening;
the open end of the hollow cannula being configured to accept the plurality of magnetic spherical beads and the non-magnetic rod.

15. The system for facilitating the treatment of lumbar degenerative disc disease of claim 10, wherein the plurality of magnetic spherical beads is configured to rearrange during motion of the specimen.

16. The system for facilitating the treatment of lumbar degenerative disc disease of claim 10, wherein the magnetic spherical beads are a suitable replacement for nucleus pulpous.
